(19) United States
(12) Patent Application Publication
(10) Pub. No.: US 2012/0226212 A1
(43) Pub. Date: Sep. 6, 2012

(54) TISSUE CONTACTING MATERIAL

(76) Inventors: Stanley Harpstead, Arden Hills, MN (US); Loren L. Barber, JR., Lake Elmo, MN (US)

(21) Appl. No.: 13/388,807

(22) PCT Filed: Jul. 30, 2010
(86) PCT No.: PCT/US10/43850

§ 371 Date: May 23, 2012

Related U.S. Application Data
(63) Continuation of application No. 12/534,938, filed on Aug. 4, 2009.

(51) Int. Cl. A61F 13/00; C08L 75/04

(52) U.S. Cl. 602/43; 602/48; 525/123

(57) ABSTRACT

A tissue contacting material comprising a plurality of regions comprising an outer region serving as a protective barrier, one or more inner regions, and a tissue contacting region. The plurality of regions each comprise one or more of a plurality of polymers selected from the group consisting of a first polymer comprising a crosslinked hydrophilic polymer and a second crosslinked polymeric matrix, formed of a crosslinkable polymer adapted to incorporate the first polymer without substantially reacting or crosslinking with the first polymer. The first and/or second polymers provide the respective regions with one or more properties including swellability in the presence of water, active agent content, permeability to the diffusion of active agent from or through the layer, moisture vapor permeability, and adhesion to tissue.

### TECHNICAL FIELD

In one aspect, the present invention relates to the field of tissue contacting materials such as wound dressings. In a related aspect, the invention relates to polymeric formulations used to provide properties such as water swellability, active agent delivery, and a moist barrier.

### BACKGROUND OF THE INVENTION

Materials for use in contacting tissue have existed in many forms and corresponding formulations, e.g., as wound dressings, cover layers for medical devices or implants, and the like. Current approaches to the treatment of wounds, for instance, include a variety of dressings, often designed to control moisture and humidity; to keep bacteria out; and to apply anti-microbial agents or growth factors. Most dressings comprise a support layer, such as a film, which may provide a barrier against bacterial or viral infection.

Wound dressings have been described that provide therapeutic agents to wounds. For example, some dressings provide a liquid permeable wound contact layer, an intermediate layer and an outer, backing layer in which one or more layers contains one or more therapeutic agents.

Burns and other related wounds such as donor tissue sites and the like present a serious problem in that they tend to produce large amounts of exudates which can cause conventional dressings to become saturated, infected or even stick to the wound. When the dressing has stuck to the wound it is extremely painful to remove and often requires surgical excision.

What is clearly needed are materials that provide an optimal combination of properties, including for instance, wettability, transparency, tissue interaction (e.g., minimal ingrowth), ease of application, the ability to be sterilized or provided in sterile form, and the like.

### BRIEF DESCRIPTION OF THE DRAWING

In the Drawings:

FIG. 1 shows a comparison of layers in a preferred embodiment of this invention, both before and after hydration, as described herein.

FIG. 2a through 2d show the relationship of various layers in a preferred embodiment of this invention.

FIG. 3 shows a plot relating to the diffusion of active agent from within a material of this invention.

### SUMMARY OF THE INVENTION

The present invention provides a tissue contacting material comprising:
a) a plurality of regions, the regions preferably comprising
   i) an outer region, generally distal to the tissue itself, and sufficient to serve as a protective barrier,
   ii) one or more inner regions directly or indirectly adjacent the outer region, preferably one or more of the inner regions containing one or more active agents, and
   iii) a tissue contacting region directly or indirectly adjacent the inner region(s), preferably containing one or more active agents,
b) the plurality of regions each comprising one or more (and preferably at least two) of a plurality of polymers selected from the group consisting of:
   i) a first polymer comprising a crosslinked hydrophilic polymer; and
   ii) a second polymer comprising a crosslinkable polymer adapted to form a polymeric matrix (e.g., heterogeneous blend) that incorporates the first polymer, e.g., in the form of an interpenetrating network, and preferably without substantially reacting or crosslinking with the first polymer,
c) the first and/or second polymers being present in forms (e.g., partially or substantially hydrated or non-hydrated), amounts, and/or ratios within the outer, inner, and tissue contacting regions, respectively, in order to provide the respective regions with one or more substantially different properties selected from the group consisting of: swellability in the presence of water, active agent content, permeability to the diffusion of active agent from or through the layer, moisture vapor permeability, and adhesion to tissue.

Applicant has discovered, inter alia, that polymeric materials can be prepared containing a blend of first and second polymers as described herein, and in turn, can be mixed in differing ways in order to provide unique physical/chemical and/or functional properties for each corresponding region (or corresponding layers within or between regions). For example, a material (including a layer thereof) can be formed using a water-based solvent in a manner that substantially retains the ability of the layer to later swell during re-hydration, in the presence of moisture and in the course of its use. By contrast, a material can be formed using a similar or equivalent polymeric blend, but substantially without the presence of water in the solvent, in order to provide a material, region or layer thereof that will provide significantly less (e.g., substantially no) swelling during re-hydration in the presence of water.

Further, those skilled in the art, given the present description, will appreciate the manner in which the use of various solvents (e.g., water-based and non-) can affect various properties of a layer or corresponding region, and in turn of the material itself, including with respect to the adherence properties to tissue and/or tensile strength of the material itself. The use of various solvents, e.g., in solvent blends as described herein, permit materials to be built by the creation of multiple layers, preferably without the use of adhesives, and without the formation of sharp transitions or edges between layers. Further the different solvent blends can be used to provide corresponding layers having respective property differences, which in turn, can be used to alter such properties as the storage capacity and diffusion properties of active agents within and/or through the various layers. Even further, the continuity (or discontinuity) that can result from a layered organization of these layers can be used to provide directional diffusion through the material itself.

### DETAILED DESCRIPTION

Accordingly, in a particularly preferred embodiment, the present invention provides a moisture vapor transmitting material (e.g., film having a plurality of layers) characterized by a continuous matrix of hydrophilic polymer (e.g., polyurethane) and crosslinked-acrylic-copolymer, within which an active agent can be stored and released in a desired (e.g., directional) fashion. Such a material can be used in any suitable manner, for instance, as a topical wound dressing, as a barrier, or a covering for use with a medical device, and the like.

One preferred material of the present invention is normally slightly translucent in the presence of an excess of water, due at least in part to the swelling of different polymers in the body of the film. However, as the material dries it tends to become more transparent, and often has the ability to remain so while in the presence of moisture vapors found in the tissue and wounds. In turn, in certain preferred embodiments, the film can again become slightly translucent, e.g., in the presence of an excess of water, for use as an indicator of the presence or volume of moisture (e.g., exudate) from the contacting tissue (e.g., wound).

In a preferred material of the present invention, the first polymer is preferably provided in the form of a crosslinked hydrophilic (co)polymer, and more preferably, a crosslinked-(meth)acrylic-copolymer. The first polymer can be provided and used in any suitable form, e.g., in the form of a powder or particles.

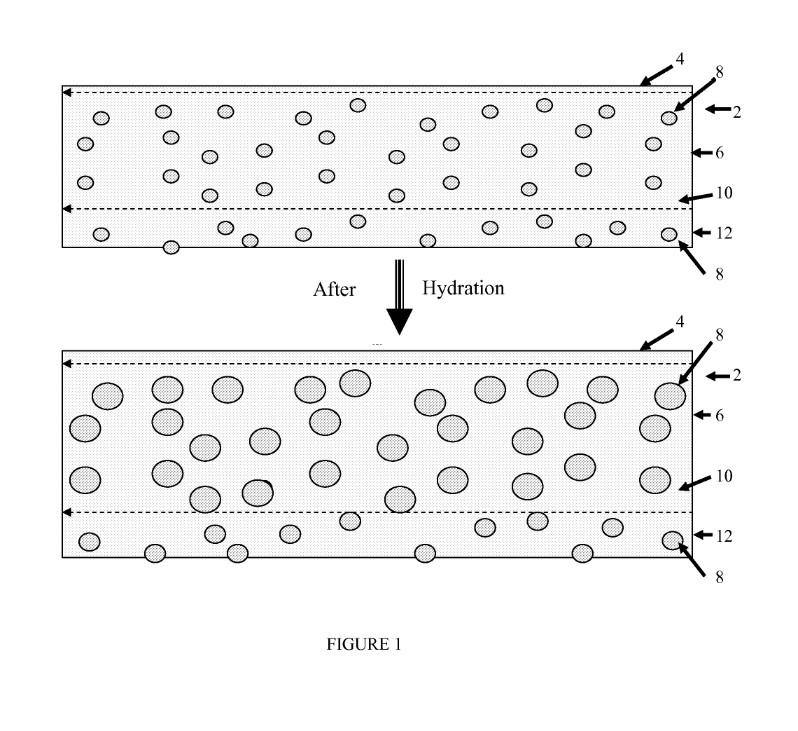

The first polymer can preferably be reversibly hydrated, and in turn swell, in the course of its use. In turn, properties such as the moisture vapor transmission of any layer (e.g., a layer within the barrier region) can be controlled by the degree of hydration of the crosslinked-acrylic-copolymer particles at the time of using the second polymer to form a polymeric matrix around the hydrated particles. FIG. 1 shows an example of tissue contacting material 2 including an outer region 4, an inner region 6 including a first polymer 8 and a second polymer 10, and a tissue contacting region 12, before hydration (in the upper film 2) and after hydration (in the lower film 2). As can be seen in the film 2 after hydration, the first polymer 8 has differentially swollen in response to hydration, with the first polymer 8 in the inner region 6 swelling more than the first polymer 8 in the tissue contacting region 12.

Those skilled in the art, given the present description, will appreciate the manner in which the moisture vapor transmission of a layer can be controlled, for instance, by adjusting the volume ratio of first to second polymers 8, 10, as well as by the respective physical-chemical characteristics of each (e.g., hydration of the first, and extent of crosslinking of the second). It is possible to adjust the size, spacing, and concentration of micro-cavities that can be formed in the resulting structure, e.g., upon partial or substantially complete dehydration of the first polymer 8 particles. For instance, larger and more numerous micro-cavities can be produced by a greater concentration and/or greater degree of hydration of the first polymer 8 particles in certain regions or layers, such as in the inner region 6. This, in turn, will typically allow for freer vapor transmission. By comparison, smaller and/or less numerous micro-cavities of the first polymer 8 in other regions or layers, such as in the tissue contacting region 12, tend to reduce the hydration potential and vapor transmission rate of the corresponding layer. The degree of cross-linking by the second polymer 10 can further be affected, for instance, by the amount of swelling of first polymer 8 particles, e.g., based upon on the amount of water, the presence of cations, and the pH of the dispersion of first polymer 8 within second polymer 10.

The greater potential for expansion of the first polymer 8 in certain regions or layers of the dehydrated film, such as the inner region 6, that can be realized during re-hydration is a function of the incomplete crosslinking of the second polymer 10 that occurred while the first polymer 8 was still hydrated. Whereas the potential for the first polymer 8 in other regions or layers, such as in the tissue contacting region 12, to expand is restricted by a more complete crosslinking of the second polymer 10. Differential rehydration of the first polymer 8 in one region or layer of the film 2 as compared to another region or layer, such as in the inner region 6 as compared to the tissue contacting region 12, differentially expands the permeability of each domain.

Crosslinked hydrophilic first polymer 8 particles are preferably provided in the form of at least partially neutralized crosslinked copolymer of monomers comprising the reaction product of at least one free-radically polymerizable carboxylic acid and at least one of an alkyl or alkaryl (meth)acrylate, wherein the at least one alkyl or alkaryl (meth)acrylate has from 11 carbon atoms to 34 carbon atoms, and optionally additional co-monomers. As used herein, the term “carboxylic acid” encompasses the corresponding conjugate base (i.e., carboxylate).

Useful free-radically polymerizable carboxylic acids have at least one carboxyl group covalently bonded to a polymerizable carbon-carbon double bond. Exemplary free-radically polymerizable carboxylic acids include, for instance, itaconic acid, (meth)acrylic acid, maleic acid, fumaric acid, salts of the foregoing, and mixtures thereof. The phrase “copolymer of monomers comprising” refers to the structure of the copolymer rather than any particular method of preparing the copolymer. For example, the copolymer may be prepared using a monomer (e.g., maleic anhydride) that on hydrolysis (before or after co-polymerization) results in a free-radically polymerizable carboxylic acid. In order to ensure good swellability of the crosslinked copolymer, the acid content typically falls in a range of from about 40 percent to about 90 percent by weight (e.g., in a range of from 50 to 70 percent by weight) of the crosslinked copolymer, although acid content values outside this range may also by used.

Useful alkyl and alkaryl (meth)acrylates have from about 11 carbon atoms to about 34 carbon atoms, and may be linear or branched. Examples of useful alkyl and alkaryl (meth)acrylates include octyl (meth)acrylate, isooctyl (meth)acrylate, octadecyl (meth)acrylate, tridecyl (meth)acrylate, and nonylphenyl acrylate. Optionally, additional co-monomers (e.g., (meth)acrylamide, butyl (meth)acrylate) may be included in the crosslinked copolymer.

Crosslinking can be accomplished by any suitable means, e.g., by inclusion of a monomer having multiple free-radically polymerizable groups (e.g., a polyfunctional monomer) in the monomer mixture prior to copolymerization, although other methods may be used. Useful polyfunctional monomers include, for example, vinyl ethers (e.g., pentaerythritol trivinyl ether, pentaerythritol tetravinyl ether, ethylene glycol divinyl ether), allyl ethers (e.g., pentaerythritol triallyl ether, pentaerythritol tetraallyl ether, ethylene glycol diallyl ether), and acrylates (e.g., 1,6-hexanediol diacrylate), and mixtures thereof. The amount of crosslinking desired typically determines the amount of polyfunctional monomer used. In order to ensure good swellability with water, the crosslink density should typically be kept at low level; for example, the value of Mc (i.e., the average molecular weight of segments between crosslinks) may be greater than about 1000 g/mole, preferably greater than about 2000 g/mole, and more preferably greater than about 3000 g/mole.

Preferably, the first polymer 8 is provided in a form (e.g., as particles) that is readily dispersible and water-swellable. This can be achieved, for instance, by the use of crosslinked-acrylic-copolymer having an average dry (substantially non-swelled) particle size in a range of from about 0.1 micrometer to about 10 micrometers, or more preferably from about 2 micrometers to about 7 micrometers, although larger and smaller particles may also be used.

Suitable crosslinked copolymers can be provided in particle sizes that are readily dispersible in a solvent solution, preferably without further cross linking to either similar particles or to other (e.g., heterogeneous) particles as the solvents evaporate. Particle sizes that are too large will tend to limit the strength of the second crosslinking copolymer; while particles that are too small will not create adequate voids in the course of dehydration, or in turn, swollen spaces during re-hydration. Applicant has discovered, inter alia, that particle sizes in the range of 0.1 micrometer to 10 micrometer are preferred, particularly when used in conjunction with a second crosslinking polyurethane copolymer. Generally, it would appear that the crosslinked polymer swells in the presence of a specific solvent solution, either due to the attraction of solvent molecules, or through the interference with internal ionic bonding due to the change in pH, or the presence of either cations or anions in the solution. This increase in size dimension preserves the potential space for the re-swelling of this copolymer particle in the presence of the swellable environment.

Typically, the first polymer 8 particles are used in the aqueous phase, in an amount from about 0.5 percent by weight to about 3 percent by weight, based on the total weight of the first polymer composition (e.g., as an emulsion, solution, or mixture), although higher and lower amounts may also be used. For example, a preferred crosslinked-acrylic-copolymer may be present in an amount in a range of from about 1 percent by weight to about 2 percent by weight, based on the total weight of the composition.

Examples of useful commercially available crosslinked-acrylic-copolymer include, for example, those marketed by Noveon, Cleveland, Ohio under the trade designations “CARBOPOL” and “PEMULEN” (e.g., “CARBOPOL 674 POLYMER”, “CARBOPOL 676 POLYMER”, “CARBOPOL 934 POLYMER”, “CARBOPOL 940 POLYMER”, “CARBOPOL 941 POLYMER”, “CARBOPOL 980 POLYMER”, “CARBOPOL 981 POLYMER”, “CARBOPOL 1342 POLYMER”, “CARBOPOL 1610 POLYMER”, “PEMULEN 1621 RESIN”, “PEMULEN 1622 RESIN”, “CARBOPOL 1623 POLYMER”, “CARBOPOL 2984 POLYMER”, and “CARBOPOL 5984 POLYMER”).

A preferred feature of the crosslinked-acrylic-copolymer marketed under the trade designations “CARBOPOL” and “PEMULEN” is that a high level of electrolytes will prevent the normal swelling of the hydrophilic portion of the molecule. Cationic species, notably sodium, often complex with the crosslinked-acrylic-copolymers and this feature can be used to alter the micro-cavities within the therapeutic films.

The first crosslinked-acrylic-copolymer can be further processed such that reactive binding sites can be incorporated into the first copolymer. These reactive binding sites can, in turn, be used to incorporate various other compounds, e.g., active agents, by means of covalent linkages. By way of example, those skilled in the art, given the present description, will appreciate the manner in which such binding sites can be further used to covalently bind proteins into the micro-cavities within the therapeutic films.

A material of the present invention further comprises a second polymer 10 comprising a crosslinkable polymer adapted to form a polymeric matrix (e.g., heterogeneous blend) that incorporates the first polymer, preferably without substantially reacting or crosslinking with the first polymer. A preferred second polymer 10 is a hydrophilic polyurethane, and more preferably, a member of the aromatic hydrophilic polyurethane family.

In order for the first polymer 8 (e.g., crosslinked-acrylic-copolymer) to be integrated into the interpenetrating network of the polyurethane matrix, the crosslinkable polymer (e.g., hydrophilic polyurethane “PU(H)”) will preferably have one or more desirable traits. It is preferred that the second polymer 10 have hydrophilic properties that permit it to remain in solution when combined with the hydrated crosslinked-acrylic-copolymer. More preferably the second polymer 10 will absorb between about 20% to about 80% of its dry weight in water, and more preferably, between about 30% to about 40% of its dry weight in water.

A second preferred trait is that the second polymer 10 (e.g., polyurethane) be sufficiently soluble in solvents that are miscible with those used to suspend the hydrated first polymer. For instance, a preferred polyurethane for use as the second soluble will be an organic solvent soluble in a polar, heterocyclic solvent, such as tetrahydrofuran (THF). More preferably the polyurethane will be soluble in a mixture of THF and a polar aprotic organic solvent such as acetone.

Given the present description, formulations of second polymers 10, e.g., aromatic hydrophilic polyurethanes, suitable for use in the present invention will be familiar to those experienced in the art, as will the use of aromatic polyether thermoplastic polyurethane compositions dissolved in solutions. Examples of polyurethanes suitable for use in the present invention include those described in U.S. Pat. Nos. 6,734,273, 5,428,123 and U.S. Pat. Pub. No. 2007/0112165, the disclosures of which are incorporated herein by reference.

Examples of suitable aromatic hydrophilic polyurethanes include Estane 58245 and Estane MVT 80AF3 TPU, which are polyether-based thermoplastic polyurethanes available from Lubrizol Advance Materials, Cleveland Ohio. Preferred polymers of this type have a durometer hardness of between about 80 A and about 50 D, and a volume swell in water of at least about 20% or more, preferably about 30% or more, and even more preferably about 40% or more. A particularly preferred second polymer 10 is available from Lubrizol as a copolymer of polyether polyurethane with polycarbonate. This copolymer had a durometer hardness of ˜80 A with a hydrated weight equal to 133% of its initial dry weight (volume swell in water of 33%). This copolymer was readily dissolvable in THF and swelled in the presence of acetone, allowing it to stay in an emulsion in the presence of water.

Preferably, the first and second polymers 8, 10 provide differing solvent solubilities, such that solvent solutions containing the first and second polymers 8, 10, respectively, can be prepared and mixed together in order to form a corresponding region having one or more desired properties selected from the group consisting of: swellability in the presence of water, active agent content, permeability to the diffusion of active agent from or through the layer, moisture vapor permeability, and adhesion to tissue. In turn, and as described herein, different regions of the film can be prepared using either the same or different polymers, forms, ratios, and/or total amounts of polymers, as well as different solvents or solvent systems, in order to provide the respective regions with desired properties that are suitably different from each other.

For example, a material (including a layer thereof) can be formed using a water-based solvent in a manner that substantially retains the ability of the material (or layer) to later swell during re-hydration, in the presence of moisture and in the course of its use. Preferably the water-based solvent blend used to make a solution that later readily re-hydrates will include a first solvent to disperse the crosslinked polymer, to minimize adherence between polymer particles; a water-based solvent that swells the crosslinked polymer particles; and a miscible partial-solvent that further interacts with the first crosslinked polymer. It is further preferable that the partial-solvent of the crosslinked polymer be a complete solvent of the second crosslinkable polymer.

More preferably the use of an agent such as propylene glycol methyl ether acetate (PMA) can be used to disperse and shield the crosslinked polymer from clumping and water is used as the water-based solvent to fully swell the crosslinked polymer. To fully swell the crosslinked polymer requires a volume of solvent sufficient to accomplish its hydration potential. In the presence of the preferred crosslinked polymer the ratio can be greater than 10× (w/v). More preferably a cationic water-based solution can be used to fully swell the crosslinked polymer.

To combine the crosslinking polymer in solution with the fully swelled crosslinked solution it is preferable to include a common solvent that is miscible with the water-based solution. In absence of this common solvent, the water based solvent can cause the crosslinking polymer to precipitate from solution and prevent the crosslinking matrix. For instance, a preferred organic solvent soluble will be a polar, heterocyclic solvent, such as tetrahydrofuran (THF) suitable for dissolving the preferred polyurethane and compatible with the water-based solvent containing the crosslinked polymer.

It is further preferred that the solvent used to dissolve a second polymer 10 such as polyurethane be quickly evaporated from the layer in the course of its formation while the water-based solvent preserve its ability to maintain the crosslinked polymer in its swollen state. If the solvent evaporates at too slow of a rate, the hydrated crosslinked polymer will tend to not maintain its desired, hydrated dimensions and the second polymer matrix will limit or reduce the dimensions of the micro-cavity hydrated space.

By contrast, a material (or layer) can be formed using a similar or equivalent polymeric blend, but substantially without the presence of water in the solvent, in order to provide a material (or layer) that will provide significantly less (e.g., substantially no) swelling during re-hydration in the presence of water. More preferably the use of propylene glycol methyl ether acetate (PMA) can be used to disperse and shield the crosslinked polymer from clumping and a THF and acetone solvent blend can be used as an emulsion of the crosslinked polymer. More preferably the polyurethane will be soluble in a mixture of THF and a polar aprotic organic solvent such as acetone.

Solvent combinations such as THF and acetone can be used in any suitable ratio, e.g., between about 3 to 1, to between about 1.5 to 1 (volume/volume). It is further preferred that the solvent (or solvent system) used to dissolve a second polymer such as polyurethane be relatively easily evaporated from the layer in the course of its formation.

Applicant has found that the absence of the non-solvent water tends to yield a film with greater adherence properties to tissue and improved tensile strength. The use of various solvents, e.g., in solvent blends as described herein, permit materials to be built by the creation of multiple layers, and preferably without the use of adhesives, and without the formation of sharp transitions or edges between layers. Further the different solvent blends can be used to provide layers having corresponding property differences, which in turn, can be used to alter such properties as the storage capacity and diffusion properties of active agents within and/or through the various layers. Even further, the continuity (or discontinuity) that can result from a layered organization of these layers can be used to provide or enhance directional diffusion through the material itself.

The solvents, partial-solvents and non-solvents of this invention are preferably relatively miscible, in order to enable the blending of both the interpenetrating copolymer network and the hydrated crosslinked polymer. Though various solvent systems can be used, the use of THF, acetone, water and PMA in the manner described herein is particularly preferred, in that it enables the creation of various layers having respective properties, while ensuring that each layer will adhere to the adjacent layers. In general the solvents used herein will preferably have various traits such as; volatility; polarity; non-toxic residuals; adequate solubility coefficient for the chosen heterogeneous co-polymers.

The active agents suitable for use in a material (or region(s) or layer(s) thereof) include drugs or other substances that can be used to treat a disease or condition in a patient or to ameliorate (e.g., treat, prevent or cure) either a disease or condition or its symptoms. In addition to drugs or other substances, such active agents can include, but are not limited to polynucleotides, polypeptides, oligionucleotides, nucleotide analogs, nucleoside analogs, polynucleic acid decoys, antibodies, chimeric antibodies, and nitric oxide releasing agents.

The active agent may take the form of any material or surface which when placed into the body causes or inhibits a reaction with a bodily substance. Patients according to the present invention are mammalian animals in need of treatment, preferably humans, including adult humans and human children. For example, an active agent incorporated into the wound dressing devices of the present invention may be used for the treatment of wounds or in promoting skin healing. The active agents can participate in, and improve, the wound healing process, and can include; antimicrobial agents, including but not limited to antifungal agents, antibacterial agents, anti-viral agents, amoebicidal agents, antifungal agents and anti-parasitic agents; anti-inflammatory agents including but not limited to antihistamines; cell growth factors; angiogenic factors; anesthetics or other pain relieving substances; protease inhibitors, mucopolysaccharides, metals; and other wound healing agents or mixtures thereof.

Therapeutic active agents can be incorporated within any of the layers of the material of the present invention. In turn, a material of the present invention can be used to provide an active agent delivery system that has good versatility and tenability in controlling the delivery of active agents. As described herein, properties such as the swellability and dimensions of each layer can be independently altered, e.g., by varying the polymer types or concentrations and/or the amount of water and other solvents present during the formation of each layer. Those skilled in the art will appreciate the manner in which such properties can, in turn, impact the kinetics and other properties of active agent availability and movement within and between layers, as well as from the material itself. As a result, these techniques can be incorporated into wound treatment barriers, or coatings on medical devices, e.g., stents, stent grafts, orthopedic devices, or tissue regeneration scaffolds, if desired.

When the active agent is a therapeutic compound, exemplary therapeutic compounds include antibiotics, antihistamines and decongestants, anti-inflammatory agents, antiparasitics, antivirals, local anesthetics, antifungal agents, amoebicidal agents, trichomonocidal agents, analgesics, antiarthritis agents, antiasthmatics, antidepressants, antidiabetics, antineoplastics, antipsychotics, neuroleptics, antihypertensives, antidepressants, hypnotics, sedatives, anxyolitic energizers, anticonvulsants, immune suppression agents, antiparkinson agents, anti-platelet agents, anti-cancer agents, muscle relaxant agents, antimalarials, blood modifiers, hormonal agents, contraceptives, sympathomimetics, diuretics, hypoglycemics, anti-coagulation agents, ophthalmics, anti-cell proliferation agents, electrolytes, diagnostic agents and cardiovascular drugs.

While not intending to be bound by theory, it would appear that the relationship between first and second polymers 8, 10 is one of an interpenetrating network (IPN), in that a polymer comprising two or more networks results from the blend of the first and second polymers 8, 10 in solution in which at least the second polymer 10 is interlaced on a molecular scale but not covalently bonded to the first polymer 8 and cannot be separated unless chemical bonds of the second polymer 10 are broken.

In a heterogeneous blend of the present invention, at least one polymer is preferably a crosslinked-acrylic-copolymer. Moisture vapor transmission, e.g., of the barrier region, can be controlled by the degree of hydration of the crosslinked-acrylic-copolymer particles, as the cross-linking interpenetrating network of the polyurethane matrix forms around the hydrated particles. The moisture vapor transmission of a layer can be determined by the volume ratio of interpenetrating polyurethane to the hydrated crosslinked-acrylic-copolymer. By controlling the amount of the polyurethane and the hydrated size of the crosslinked-acrylic-copolymer, it is possible to regulate the size of the micro-cavities in the polymer. Larger micro-cavities, produced by a greater degree of hydration, allow for freer vapor transmission, whereas smaller micro-cavities reduce the hydration potential and vapor transmission rate.

In a preferred embodiment of the present invention, delivery of the desired agents may be controlled, for instance, by the movement of liquid through the hydrated matrix. Though not wishing to be bound by any theory, it is preferred that the active agent incorporated within a matrix of the present invention will be unbound and entrapped in the heterogeneous polymer mass, e.g., after solvents and water have been substantially eliminated from the films during drying. When the film is re-hydrated, by absorbing water vapor from the tissue or exudates from the wound, it is thought that the free liquid portion of the hydrated polymer matrix acts as a solvent and as a means to deliver desired active agents.

The ability of the agent to move freely throughout the matrix in the free liquid phase is particularly preferred for use of the agent delivery system of the present invention. When the agent is dissolved in the free liquid phase, a concentration gradient of the active agent can be created between the matrix of a wound dressing device and the moisture of the wound itself. Therefore, when the matrix is placed onto a moist surface such as an open wound, the soluble agent will move through the free liquid phase toward the agent-free wound moisture, resulting in the delivery of the agent to the wound. This movement of soluble agent further upsets the equilibrium between soluble and insoluble agents, and causes more active agent to dissolve into the free liquid phase, thus causing additional agent to be delivered to the wound. In embodiments in which the desired agent is incorporated directly into the matrix, rather than into other delivery vehicles, such as liposomes, the agent may be dissolved in the free liquid phase and reliably delivered to the wound through the process described above.

Delivery of the desired agents can also be controlled by the degree of cross-linking in the interpenetrating polyurethane matrix that is allowed based on the ratio of the first polymer 8 (crosslinked-acrylic-copolymer) to the second polymer 10 (hydrophilic polyurethane). The degree of polyurethane cross-linking is further controlled by the amount of swelling/hydration the crosslinked-acrylic-copolymer is allowed to experience based on the amount of water and/or the pH of the dispersion. By controlling the amount of the polyurethane and the hydrated size of the crosslinked-acrylic-copolymer, it is possible to regulate the size of the micro-cavities in the polymer blend. Larger micro-cavities, produced by a greater degree of hydration, allow for freer migration and quicker delivery of the desired agent, whereas smaller micro-cavities increase the delivery time.

In other embodiments, e.g., where the directional delivery of low molecular weight agents is desired, the hydrated micro-cavities of the outer layer can be further restricted by the incorporation of a non-hydrophilic (e.g. hydrophobic) polyurethane into the cross-linking interpenetrating network.

In wound therapies the ability to adhere the polymeric film tightly to the healing wound is complicated by large quantities of exudates. Frequently this process requires the addition of pressure sensitive adhesive that alters the vapor transfer away from the wound or the diffusion properties of the active agent toward the healing tissue. Accordingly, the present invention can provide a wound dressing material comprising a moisture film with the inherent property of a surface that is tacky to moist tissue.

In a particularly preferred embodiment, a material of the present invention is used as a wound dressing, in a manner that provides restoration of anatomic continuity, structure, function and appearance. Over an extended time the wound is remodeled to reduce scar quantity while increasing strength and quality. A wound dressing that comprises a material of this invention provides for the possibility of a single wound healing modality (barrier or covering) that can be used to provide progressive care through the healing process for all wound types. During this healing sequence, the material provides opportunities for the medical practitioner to monitor, manage and influence the wound healing process, based on the use of a wound covering having broad applications throughout the healing process. In turn, a transparent barrier film of this invention allows the practitioner to monitor the presence of infection and that can reduce the risk of infection or treat existing infections, as well as to reduce pain, and minimize inflammation.

With proper preparation and maintenance of the wound bed, a covering of this invention can reduce bioburden, protect the wound from further injury, and augment the healing environment, lessening the chance that patients will suffer poor outcomes or are forced to live with chronic, complex wounds or severe scarring. Further the time for achieving the wound healing process can be decreased, in turn, decreasing the cost and increasing patient comfort.

Moreover, and particularly with regard to chronic wounds, such as venous ulcers, pressure ulcers and diabetic ulcers, the ability to visually monitor the healing wound and transfer the increased moisture generated are essential to the use of transparent barrier films of this invention.

Applicant has discovered a method to make a barrier film with a wound facing adherent surface, significant vapor permeability that avoids the use of apertures or fibrous materials, and that offers the potential for containing active agents with a diffusion profile directed toward the wound. In a material of this invention the material's tackiness can arise from the combination of first and second polymers, e.g., crosslinked-acrylic-copolymer (PAA) incorporated within the polyurethane interpenetrating matrix. In turn, the material can be easily removed from the tissue in the presence of an excess of water or saline, but will maintain its apposed location, while even following the papillary ridges of the skin.

Preferred materials of the invention have a desirable soft surface feel when they are used in the manufacture of articles for bodily contact. Further the process allows for the formation of articles of various sizes and shapes to accommodate the desired area of treatment.

Polymeric materials can also be used to facilitate the transfer of fluids and the diffusion of the active agents. The polymeric film in this invention can serve as a repository for the active agents to be delivered for therapeutic healing of a wound. As used herein, the term active agent refers to a therapeutic, palliative, or diagnostic agent used in wound healing or other disease procedures. Such active agents can be incorporated into the polymers and/or heterogeneous polymer solutions used in the invention; or dispersed within the crosslinked-acrylic-copolymer; or polymer matrix; or coated on the surface of layer during the process of forming a film; or placed on the surface of the film prior to clinical use as a wound dressing; or soaked into the film prior to placement; or covalently incorporated, for instance, onto the crosslinked-acrylic-copolymer, or onto the polyurethane interpenetrating matrix; or incorporated into the coating of an implantable surgical device having different therapeutic aims as will be appreciated by those skilled in the art, given the present description. Such approaches are particularly preferred in embodiments where release of the active agent from the film is desirable, for example, by contact with a tissue surface, or with blood borne cells or serum after insertion of the film or a device coated with the film into the body.

In certain embodiments, a therapeutic active agent may be present in the film in particulate or soluble or otherwise dispersible form, so that it can pass out of the film into the wound once the hydrated channels are formed by the action of interstitial fluid or wound exudates. In other embodiments, the therapeutic agent may be retained inside the film envelope even after the channel has opened, for example by being dispersed in or on a substrate that is too large to fit through the channel or by being covalently bonded within or on to the polymer matrix. An example would be a silver treated cloth.

In certain embodiments, a therapeutic active agent can be dispersed in or on particles suitable for drug delivery, and the particles in turn may be incorporated into the therapeutic film. The particles can be made by any suitable technique, including comminution, coacervation, or two-phase systems for example as described in U.S. Pat. No. 3,886,084. Techniques for the preparation of medicated microspheres for drug delivery are reviewed, for example, in Polymeric Nanoparticles and Microspheres, Guiot and Couvreur eds., CRC Press (1986). The microparticles are preferably loaded with from about 1 to about 90 wt. %, and more preferably from about 3 to about 50 wt. % of the active agent.

Preferred embodiments of the present invention however, address the need for a less expensive, quicker, and more reliable method for incorporating a wider range of desired active agents into wound dressing devices. Preferred embodiments also provide a means to control the release of the desired agents over time via facilitation or restriction of water movement through the polymer matrix and the degree of cross-linking in the matrix and manipulation of active agent concentration. In a preferred embodiment, given the significant presence of H₂O in the co-polymer dispersions used to form the individual layers, the desired active agents may be directly incorporated into the matrix by adding the agents into the initial formulation for the matrix prior to cross-linking. This method of incorporation is inexpensive, rapid and reliable, and most surprisingly, the incorporated agents are not affected by the process of polymerization and retain their biological activities.

In a preferred embodiment, a material of this invention can be prepared as a polymeric film comprising a heterogeneous (e.g., phase separated) blend of two or more polymers. The film dimensions and functions can be altered by using multiple layers of heterogeneous blends or single polymers. When the multiple layers in a film are created by using the same heterogeneous polymer blends dissolved in compatible solvent blends the use of adhesive or other techniques to adhere the respective layers is avoided. Altering the ratios of the heterogeneous polymers in the polymer blend will change the structural and functional properties of the individual layers in the finished barrier film. Further, altering the ratio of solvents, partial solvents and non-solvents within the multiple layers during the formation of the film varies the vapor transfer and swellability of the finished barrier film in the presence of water. These parameters can all be adjusted to achieve valuable therapeutic traits in a transparent barrier film. Finally, the use of water among the non-solvents among one or more of the multiple layers during the formation of the barrier film creates potential swellable spaces for use in storage and delivery of active agents.

In turn, a material of this invention minimizes or avoids altogether limitations that exist with conventional laminate materials, e.g., structural failure due to cracking and delamination of the multiple layers, or limitations in the range and/or amounts of active agents that can be included with a delivery system, and the range of rates at which the included active agents are delivered therefrom. Rather than the conventional use of a single polymer to form the layers within the barrier film, a material of the present invention involves the use of a uniform selection of polymers within each layer and the functional properties of each layer are dependent on the different solvent/non-solvent platforms with differing swellability used during the condensation process. This process allows for films with different functioning layers to be formed without adhesives, mechanical or heat processes to bond layers.

Those skilled in the art, given the present description, will understand the manner in which a dressing of this invention can be prepared using any suitable techniques. Preferably, the dressing is prepared using a plurality of polymers, as described herein that are combined in corresponding total amounts and ratios using solvents that can themselves be used in varying amounts and combinations to provide the outer, inner, and tissue contacting regions 4, 6, 12, respectively. Certain preferred properties (e.g., transparency, hydration, adherence to skin) are manifest in the course of preparing the dressing, while other preferred properties (e.g., swellability potential, moisture vapor transmission, storage and availability of active agent) might be manifest once the solvents have been substantially removed, resulting in a dressing that can be used or packaged for use having an optimal combination of properties as described herein.

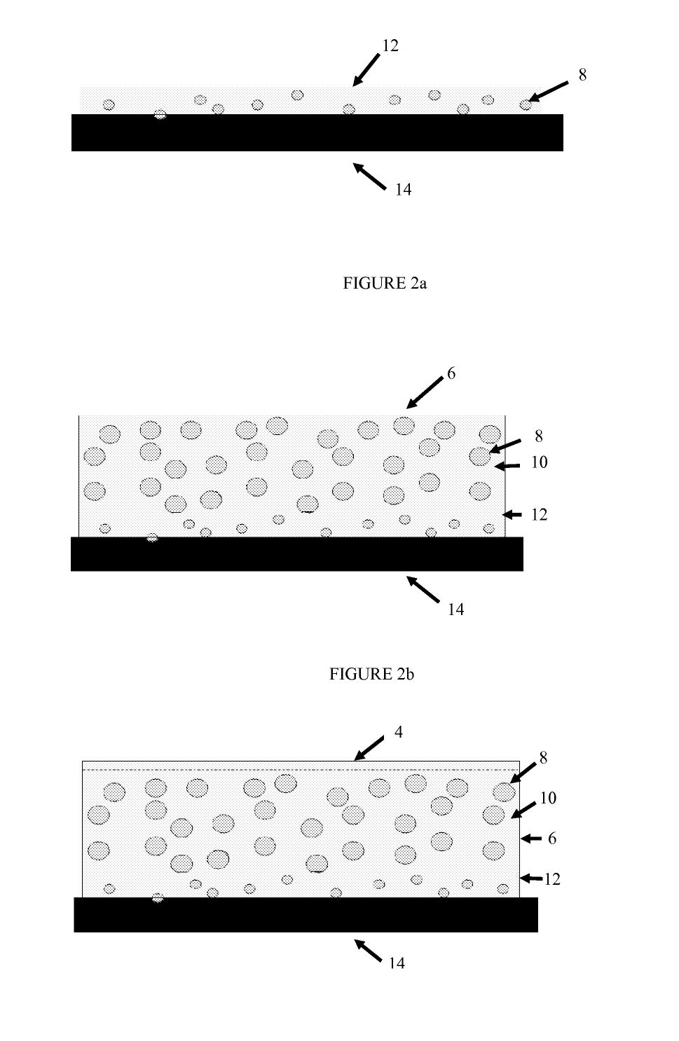

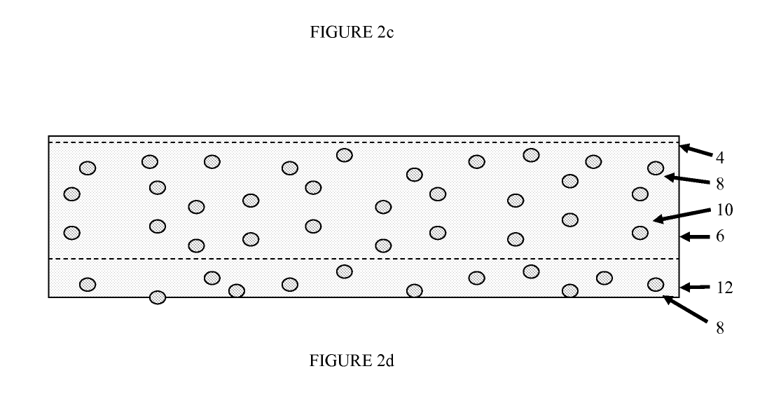

The dressing can be prepared in any suitable manner, as will become apparent to those skilled in the art, e.g., by coating or casting solutions sequentially and in any desired order, to form the corresponding regions. The membrane is formed by sequential coating of polymer solutions on a suitable release surface 14 (e.g. glass surface) as shown, for example, in FIGS. 2a-2d. Each layer is dried before the subsequent layer is deposited. The polymer solutions are made by methods familiar with the art. In many cases the sequence of dissolving polymers, combining solutions and agitation methods can alter the specific ratios and are included in these methods.

For instance, in one preferred embodiment, a tissue contacting region 12 is formed first. In such an embodiment, a suitable (e.g., coating) solution can be prepared by the combination of a first solution comprising a first polymer 8 in a solvent system that comprises, for instance, THF:PMA:acetone (e.g., in a volume ratio of 60:5:35), with a second solution comprising the second polymer 10 in a solvent solution that comprises, for instance, THF as the solvent. By way of example, the first polymer 8 can first be dispersed in a suitable solvent such as PMA alone, thereby wetting the polymer, after which the dispersed polymer solution can be combined under agitation with the THF and acetone combination in order to prepare an emulsion. In turn, the second polymer 10 is itself pre-dissolved in THF.

The first and second solvent solutions can be combined under agitating conditions and in a manner suitable to form a stable emulsion. Layers of this film can be deposited on a release substrate in such a manner that they form a continuous film and the solvents allowed to evaporate, as shown in FIG. 2a, for example.

Various parameters, including the relative amounts of both first and second polymer-containing solvent solutions, can be chosen and adjusted to provide an optimal combination of desired properties, including miscibility of the solutions themselves, and ultimately, improved adherence to tissue. Typically, for instance, the first and second polymers 8, 10 will be present in a final (dry weight) ratio of between 40:60 and 60:40. In turn, first and second solutions will be combined in volume ratio of about 85 to about 15 respectively. Optionally, the tissue contacting region 12 can include active agent, for instance, of the type and in the manner described herein.

In turn, one or more inner region(s) 6 can be formed upon a tissue contacting region 12 by utilizing solutions wherein the first polymer 8 is in a hydrated state. In such an embodiment, a suitable (e.g., coating) solution can be prepared by the combination of a first solution comprising a first polymer 8 in a solvent system that comprises, for instance, PMA:H₂O:THF (e.g., in a volume ratio of 15:60:12), with a second solution comprising the second polymer 10 in a solvent solution that comprises, for instance, THF as the solvent. By way of example, the first polymer 8 can first be dispersed in a suitable solvent such as PMA alone, thereby wetting the polymer, after which the dispersed polymer solution can be combined under agitation with the H₂O and THF combination in order to prepare an emulsion. In turn, the second polymer 10 is itself pre-dissolved in THF.

The first and second solvent solutions can be combined under agitating conditions and in a manner suitable to form a stable emulsion. Layers of this film can be deposited on the tissue contacting region in such a manner that they form a continuous film and the solvents allowed to evaporate, as shown in FIG. 2b, for example. The THF in the inner region solutions can be sufficient to dissolve the surface of the tissue contact region to allow the second polymer 10 to crosslink between layers thereby firmly adhering the adjacent layers.

Various parameters, including the relative amounts of both first and second polymer-containing solvent solutions, can be chosen and adjusted to provide an optimal combination of desired properties, including miscibility of the solutions themselves, and ultimately, improved parameters of the inner layer. Typically, for instance, the first and second polymers 8, 10 will be present in a final (dry weight) ratio of between 40:60 and 60:40. In turn, first and second solutions will be combined with THF in volume ratio of about 68:10:22 respectively (solution 1:solution 2:THF). Optionally, the inner layer region can include active agent, for instance, of the type and in the manner described herein.

In a preferred embodiment, active agent is provided in a form selected from the group consisting of a) present in the first polymer solution, b) present in the second polymer solution, c) added to the combination of first and second polymer solutions, and/or d) added as a separate layer above or below the region.

Finally, an outer region 4 can be formed upon the outermost inner region as shown, for example, in FIG. 2c. In turn, one or more outer region(s) can be formed upon a inner region by utilizing solutions wherein the second polymer 10 is pre-dissolved in THF. The completed film is shown in FIG. 2d after all solvents including water have evaporated and the film has been removed from the release surface 14.

Layers of this film can be deposited on the inner region 6 in such a manner that they form a continuous film and the solvent allowed to evaporate. The THF in the outer region solution can be sufficient to dissolve the surface of the inner region 6 to allow the second polymer 10 to crosslink between layers thereby firmly adhering the adjacent layers.

Various parameters, including the relative amounts of second polymer-containing solvent solution, or the incorporation of a hydrophobic polymer, can be chosen and adjusted to provide an optimal combination of desired properties, including miscibility of the solutions themselves, and ultimately, improved structural integrity of the final barrier film and directed diffusion of the active agent. Typically, for instance, the second polymers 10 in solution will be present in a final (dry weight) ratio of between about 4% and about 6%.

### EXAMPLES

### Example 1

The crosslinked-acrylic-copolymer solution containing Pemulen 1622—Lubrizol, Cleveland, Ohio (1.69% solids) is made by first “wetting” 1.0 part of crosslinked-acrylic-copolymer (Pemulen 1622) with 9.0 parts of propylene glycol methyl ether acetate (PMA). In a separate container 40 parts of H₂O are combined with 9 parts THF. The two solutions are combined while vigorously shaking. The crosslinked-acrylic-copolymer will swell in the presence of H₂O and stay in solution without forming large clumps.

An inner region is formed by use of a H₂O/THF dispersion containing hydrophilic polyurethane “PU(H)” (Lubrizol, Cleveland, Ohio—polyether w/poly carbonate, ˜80 A durometer engineering run HP-4080A-20, DC-01-61, with a hydrated weight of 133% of initial dry weight) and poly acrylic acid (Pemulen 1622—Lubersol, Cleveland, Ohio). To make the dispersion containing both the hydrophilic polyurethane and crosslinked-acrylic-copolymer, a sequence of steps is required. The hydrophilic polyurethane is first dissolved by combining 0.75 parts PU(H) into 17.25 parts THF. After fully dissolving the PU(H) add, 38 parts of the crosslinked-acrylic-copolymer Base Solution. The end result of this process will be the formation of a dispersion with 1.34% PU(H) and 1.15% 1622 or a 53.8%:46.2% ratio. Further this solution has ˜46% H₂O and this property can be used to carry a water soluble agent into the film, either through the coating process or be adding it to a dry layer during the film formation. Tests of this inner region solution in isolation indicate that it will swell to 533.3% of its dry weight in the presence of H₂O.

The tissue contacting region (“H₂O-less” solution of crosslinked-acrylic-copolymer and PU(H)) is formed by first combining 20.0 parts THF with 3.0 parts of acetone. To this solution 4 parts of a 12.5% solution of PU(H) dissolved in THF. After vigorous mixing/shaking 4 parts of 10% Pemulen 1622—CROSSLINKED-ACRYLIC-COPOLYMER (in PMA) solution. Finally, 0.5 parts of H₂O are added for the solids to completely go into solution. Note: This solution forms a 1.59% PU(H) solids and 1.27% crosslinked-acrylic-copolymer solids solution or a 55.5%:44.5% ratio that contains ˜1.6% H₂O. This solution is very tacky to skin when slightly wetted. Tests of this tissue contacting region solution in isolation indicate that it will swell to 122.5% of its dry weight in the presence of H₂O.

The outer region solution is a 4% solution of the hydrophilic polyurethane dissolved into THF. Tests of this outer region solution in isolation indicate that it will swell to 130% of its weight in the presence of H₂O.

The coating process in this example comprised a 2.5 mil thickness of the tissue contacting material, three coats of 10.0 mil thickness of the inner region, finished with a single 5.0 mil thickness of the 4% PU(H) outer region solution. The dried membrane is then fully wetted with dH₂O and gently lifted from the glass surface. The membrane is transferred to a Teflon sheet for further handling and allowed to dry before attaching to a structural edge.

Samples from this formed film were evaluated for moisture vapor transmission rate (MVTR—37° C., 20% RF/100% RF). The test utilized the upright cup method. Barrier films were wetted and placed over upright cups filled with dH₂O. The films were clamped in place and allowed to equilibrate for 30 minutes at 37° C. in an oven. The cups were weighed at time zero (T0) and again after 2 hours (T2), 5 hours (T5) and 21 hours (T21). The rate of moisture evaporation is calculated by measuring the weight lost and dividing by the area of exposed film. The rate is expressed as grams lost/m²/24 hours. The barrier films MVTR was 9,563.1±282.6 gm/m²/24 hr. (Note: a control of open cups had a weight loss of 7,265.9±266.7 gm/m²/24 hr.)

### Example 2

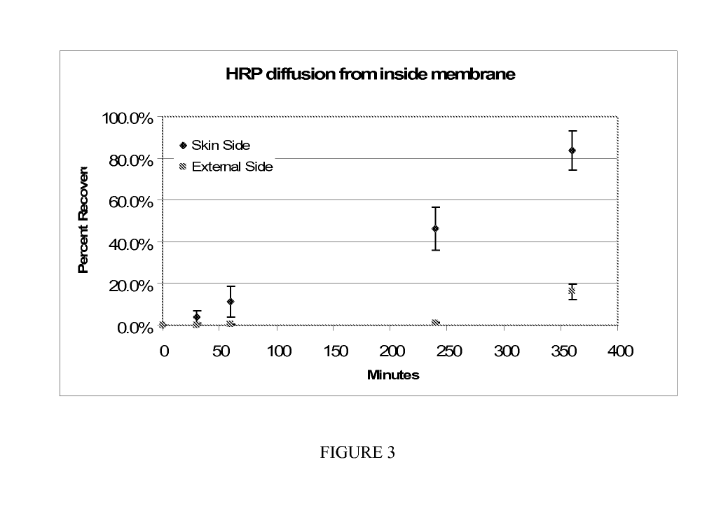

In a second example the films were made in a similar fashion, but 5 μl drops of HRP, the enzyme peroxidase from horseradish (5 mg/ml—Molecular Probes; P-917), were spatially placed after the second inner region layer had dried. After these HRP spots were dried over night, the other layers were added and the membrane removed with dH₂O. The spots were placed over the opening in a diffusion chamber and both sides of the film were filled with a PBS solution. Samples (40 μl) were taken at T0, T30, T60, T240 and T360 minutes. The samples were measured for HRP activity using SigmaFast OPD #P9187 and read using an absorbance meter. The results are shown in FIG. 3, where the diamond shaped dots (upper group of dots) represent percent HRP diffusion from the skin side or Tissue Contacting side of the film, and the square shaped (lower group) of dots represent percent HRP diffusion from the external side of the film. The results indicate that the HRP is still active and functional and also that the enzyme preferentially diffused toward the Tissue Contacting side of the barrier film. The ratio of HRP diffusion to the tissue side was 5:1 as compared to the external side.

The resulting layer thicknesses within the films created by this process are:

| | |
|---|---|
| tissue contacting region = | 1.8 μm |
| inner region (3 layers) = | 18.6 μm |
| external outer region = | 4.8 μm |
| Total = | 25.3 μm |

Hence, it can be seen that a preferred material of this invention can include three regions, including an outer region 4 with minimal permeability, an inner region 6 with greatest permeability and hydration potential for storage and delivery of pharmaceutical agents, and a tissue contacting region 12 with “tunable” permeability to alter agent kinetics.

What is claimed is:

1. A tissue contacting material comprising:
a) a plurality of regions comprising
i) an outer region, generally distal to the tissue itself, and sufficient to serve as a protective barrier,
ii) one or more inner regions directly or indirectly adjacent the outer region, and
iii) a tissue contacting region directly or indirectly adjacent the inner region(s),
b) the plurality of regions each comprising one or more of a plurality of polymers selected from the group consisting of:
i) a first polymer comprising a crosslinked hydrophilic polymer; and
ii) a second crosslinked polymeric matrix, formed of a crosslinkable polymer adapted to incorporate the first polymer without substantially reacting or crosslinking with the first polymer,
c) the first and/or second polymers being present in respective forms, amounts, and/or ratios within the outer, inner, and tissue contacting regions, respectively, in order to provide the respective regions with one or more substantially different properties selected from the group consisting of swellability in the presence of water, active agent content, permeability to the diffusion of active agent from or through the layer, moisture vapor permeability, and adhesion to tissue.

2. A materials according to claim 1, wherein the first polymer comprises a crosslinked (meth)acrylic copolymer.

3. A material according to claim 1 wherein the second polymer used to form the matrix comprises a crosslinkable hydrophilic polyurethane.

4. A material according to claim 1 wherein the first polymer is present in a substantially dehydrated state, sufficient to create corresponding microcavities within the polymeric matrix.

5. A material according to claim 4 wherein the active agent is substantially incorporated in the microcavities formed upon dehydration of the first polymer.

6. A material according to claim 1 wherein the first polymer is present in a substantially dehydrated state and is constrained from rehydration by the second crosslinked polymeric matrix.

7. A material according to claim 1 wherein the first polymer is present in a substantially dehydrated state and is rehydratable without substantial constraint imposed by the second crosslinked polymeric matrix.

8. A material according to claim 1, wherein the material incorporates one or more active agents.

9. A material according to claim 1 wherein the active agent is substantially incorporated in the matrix formed by the second polymer.

10. A method of preparing a material according to claim 1, the method comprising the steps of:
a) providing the first and second polymers in respective solvent solutions, sufficient to hydrate the first polymer and suspend the second polymer,
b) the first and second solutions being sufficiently miscible in order to permit the second polymer to be substantially crosslinked while containing the first polymer in a hydrated state, and
c) the first and second solutions being removed in order to dehydrate the first polymer, leaving microcavities in the second polymer matrix.

11. A wound healing composition comprising a material according to claim 1.

12. An implant material comprising a material according to claim 1.
